(19) United States
(12) Patent Application Publication
(10) Pub. No.: US 2024/0148034 A1
(43) Pub. Date: May 9, 2024

(54) CONSTRUCTS COMPRISING FIBRIN OR OTHER BLOOD PRODUCTS FOR MEAT CULTIVATION AND OTHER APPLICATIONS

(71) Applicant: Terasaki Institute for Biomedical Innovation, Los Angeles, CA (US)

(72) Inventors: Seyedvahid Hosseini, Newbury Park, CA (US); Alireza Khademhosseini, Los Angeles, CA (US); Mohammad Ali Darabi, Los Angeles, CA (US); Benjamin Zamanian, Los Angeles, CA (US); Seyed Rasoul Seyed Mahmoud, Olathe, KS (US)

(73) Assignee: Terasaki Institute for Biomedical Innovation, Los Angeles, CA (US)

(21) Appl. No.: 18/280,994

(22) PCT Filed: Mar. 9, 2022
(86) PCT No.: PCT/US2022/019594

§ 371 Date: Sep. 8, 2023

Related U.S. Application Data
(60) Provisional application No. 63/314,171, filed on Feb. 25, 2022.
(60) Provisional application No. 63/314,191, filed on Feb. 25, 2022.
(60) Provisional application No. 63/300,577, filed on Jan. 18, 2022.
(60) Provisional application No. 63/279,617, filed on Nov. 15, 2021.
(60) Provisional application No. 63/279,631, filed on Nov. 15, 2021.
(60) Provisional application No. 63/279,642, filed on Nov. 15, 2021.
(60) Provisional application No. 63/279,644, filed on Nov. 15, 2021.
(60) Provisional application No. 63/164,397, filed on Mar. 22, 2021.
(60) Provisional application No. 63/164,387, filed on Mar. 22, 2021.
(60) Provisional application No. 63/159,403, filed on Mar. 10, 2021.

(51) Int. Cl. A23L 13/40

(52) U.S. Cl. CPC A23L 13/424

(57) ABSTRACT

The present disclosure generally relates, in certain aspects, to cultivated meat and other cultivated animal-derived products. In some embodiments, muscle and/or fat cells can be grown on microcarriers or other scaffolds, for example, in a bioreactor or other in vitro cell culture system. The microcarriers or other scaffolds can comprise materials such as fibrin. The fibrin may be formed into hydrogels or other articles, which may be edible in some cases. The microcarriers may also contain grooves or other structures in some instances. In certain embodiments, the microcarriers may be present within the final product, e.g., in a cultivated meat product. Other embodiments are generally directed to methods of making or using microcarriers or cultivated meat products, kits involving these, or the like.

### RELATED APPLICATIONS

This application claims the benefit of U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; U.S. Provisional Patent Application Ser. No. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.” Each of these is incorporated herein by reference in its entirety.

### FIELD

The present disclosure generally relates to cultivated meat and other cultivated animal-derived products.

### BACKGROUND

Cultivated meat, or cell-based meat, is meat that is produced using in vitro cell culture or bioreactors, instead of being harvested from live animals. In many cases, the meat that is produced may include muscle cells and fat cells. Such meat may include, for example, chicken, beef, pork, or fish. Such technologies have the potential to revolutionize agriculture, for example, by decreasing the amount of land necessary to produce meat, avoiding unethical farming of animals, or increasing the available food supply. However, it is still difficult and expensive to culture cells for applications such as cultivated meat, and thus improvements are needed.

### SUMMARY

The present disclosure generally relates to cultivated meat and other applications. The subject matter of the present disclosure involves, in some cases, interrelated products, alternative solutions to a particular problem, and/or a plurality of different uses of one or more systems and/or articles.

In accordance with certain embodiments, to produce a cultivated meat product, myoblasts may be caused to proliferate and/or fuse, and in some cases, form a higher order construct, myotubes. Certain embodiments, for instance, are directed to strategies for microcarriers that may allow proliferation and/or differentiation of myoblasts to myotubes, e.g., in suspension bioreactors, and/or in other reactors including those described herein.

For example, in one set of embodiments, fibrin-based constructs are used. These can be produced, in some embodiments, using molding or extrusion techniques to produce certain shapes such as microfibers, micro-whiskers, micro-flakes, etc. In certain embodiments, the microcarriers are based on animal-based proteins. In some cases, microfibers such as these may contain grooves, for example, as a secondary hierarchical architecture, which may be used to promote alignment during the growth of the myoblasts on the microcarrier. Microcarriers such as those described herein may allow cell proliferation and/or differentiation of myoblasts to myotubes, e.g., without additional steps in some cases. In certain embodiments, such techniques may be used to produce the final product, for example, a microcarrier may be used in the final product, e.g., a cultivated meat product. In addition, in some cases, the microcarriers may be combined with a fat replica, for example, comprising a fat emulsion and a hydrogel, to produce a cultivated meat product, or another cell-based animal-derived product.

In some embodiments, the cultivated meat may be grown in a bioreactor comprising a cell culture media that is, at least partially, comprised of blood products. The blood products may be harvested from a human or non-human and may comprise whole blood or blood components such as platelet rich plasma (PRP), platelet poor plasma (referred to as plasma), a platelet concentrate, a lysate of red blood cells, a platelet lysate (PL), growth factors, proteins, cytokines, or the like. In another embodiment, the non-human blood plasma may be used as a nutrient source in a bioreactor. In certain embodiments, the serum is fetal bovine serum. In some cases, the blood may be obtained from commercial vendors. However, in some embodiments, the non-human blood plasma may be obtained from living animal donors.

In addition, one aspect is generally directed to a cultivated meat product. In one set of embodiments, the cultivated meat product comprises microcarriers comprising fibrin and non-human animal cells.

Another aspect is generally directed to a method. According to one set of embodiments, the method comprises fabricating microcarriers comprising a hydrogel comprising fibrin, and culturing non-human cells on the microcarriers.

In another set of embodiments, the method comprises lysing non-human red blood cells to produce a cell lysate, and mixing the cell lysate and non-human muscle cells on microcarriers comprising fibrin to produce a tissue mass of at least 10 g.

In another aspect, the present disclosure encompasses methods of making one or more of the embodiments described herein, for example, cultivated meat. In still another aspect, the present disclosure encompasses methods of using one or more of the embodiments described herein, for example, cultivated meat.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a process of milling fibrin hydrogels to form fibrin microcarriers, in accordance with one embodiment;

FIG. 2A illustrates optical images of the fibrin microcarriers following milling for 40 seconds, 80 seconds, or 110 seconds, in other embodiments;

FIG. 2B illustrates the average diameter of fibrin microcarriers following milling for 40 seconds, 80 seconds, or 110 seconds, according to yet other embodiments;

FIG. 3 illustrates the concentration of myoblast cells as a function of time for cells the surface of milled fibrin microcarriers, in still another embodiment;

FIG. 4A illustrates the cell viability of myoblasts encapsulated in fibrin microcarriers, post milling, following 3 days, 5 days, and 7 days of cell culture, using calcein-AM as a fluorescent biomarker for cell viability, in accordance with yet another embodiment;

FIG. 4B illustrates the quantification of the fluorescent signal from FIG. 4A using standard imaging techniques and normalized to the two dimensional projection of the microcarrier surface, in another embodiment;

FIG. 5 illustrates the concentration of myoblast cells as a function of time for cells cultured on fibrin microcarriers fabricated under different conditions, using non-human blood plasma prepared at 1.1 mg/mL, 4.4 mg/mL, or 8.8 mg/mL, in still another embodiment;

FIG. 6 illustrates a scanning electron micrograph of fibrin microfibers fabricated by electrospinning solutions of non-human blood plasma, in yet another embodiment;

FIGS. 7A-7B illustrate the cell viability of primary bovine satellite cells seeded on the surface of fibrin microfibers fabricated by electrospinning, according to still another embodiment.

### DETAILED DESCRIPTION

The present disclosure generally relates, in certain aspects, to cultivated meat and other cell-based animal-derived products. In some embodiments, muscle and/or fat cells can be grown on microcarriers or other scaffolds, for example, in a bioreactor or other in vitro cell culture system. The microcarriers or other scaffolds can comprise materials such as fibrin. The fibrin may be formed into hydrogels or other articles, which may be edible in some cases. The microcarriers may also contain grooves or other structures in some instances. In certain embodiments, the microcarriers may be present within the final product, e.g., in a cultivated meat product. Other embodiments are generally directed to methods of making or using microcarriers or cultivated meat products, kits involving these, or the like.

Cultivated meat is often described using terms such as cultured meat, tissue mass, cellular (or cell-based) meat, slaughter-free meat, and synthetic meat, among other related terms. One aspect of the present disclosure is generally directed to a cultivated meat product that includes muscle cells that are cultivated or cultured, e.g., on microcarriers comprising fibrin, or other suitable scaffolds or microcarriers. Cultivated meat products are typically produced using in vitro cell culture or bioreactors, as opposed to “regular” meat that is grown and harvested from live animals. Myoblasts can be seeded on scaffolds or microcarriers and allowed to grow, e.g., in a cell culture system. The myoblasts can fuse together to form myotubes, which are the foundation of muscle fibers and meat in general. However, in [symbol] cell culture, the myoblasts do not adhere strongly, which results in poor proliferation. In addition, two-dimensional cell culture constructs lack the hierarchical structure that is characteristic of native muscle. Such random myoblast structures do not adequately form muscle fibers, and are often a poor meat substitute.

Thus, some embodiments, as described herein, are generally directed to microcarriers or scaffolds comprising fibrin. Fibrin is an edible fibrous protein involved in the clotting of blood. It can be formed, for example, by the action of the protease inhibitor thrombin on fibrinogen, which causes it to polymerize and form a clot. Fibrin can be used as a passive scaffolding material in some embodiments. However, in some embodiments, fibrin can specifically bind certain growth factors in the cell culture media that promote cell adhesion, proliferation, and migration. Non-limiting examples include fibronectin, hyaluronic acid, von Willebrand factor, or the like.

In certain embodiments, microcarriers or scaffolds such as those discussed herein may be treated to facilitate binding of cells, such as myoblasts. For example, the microcarriers or scaffolds may be exposed to non-human serum, which may include growth factors that bind to the microcarriers or scaffolds. The growth factors may, for example, promote cell adhesion, proliferation, and/or migration of cells into the microcarriers or scaffolds. In addition, in some cases, the microcarriers or scaffolds may have structures, such as grooves, that may allow the cells such as myoblasts to become aligned in a specific direction, although this is not a requirement. Such structures are described in U.S. Ser. No. 63/159,403, filed Mar. 10, 2021, entitled “Constructs for Meat Cultivation and Other Applications,” by Khademhosseini, et al., incorporated herein by reference in its entirety.

In some embodiments, the microcarriers or scaffolds may comprise any material that forms an edible hydrogel, such as fibrin. For example, in one embodiment, a microcarrier may be formed from a non-human blood plasma, or platelet rich plasma (PRP), both of which contain plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma, and/or PRP, containing fibrinogen, e.g., at least 10 wt %, or more in some cases.

In certain embodiments, non-human cells such as myoblasts may be seeded on the microcarriers or other scaffolds, and grown in a bioreactor or other in vitro cell culture system. For instance, myoblasts may be grown on microcarriers and, in some embodiments, allowed to differentiate or fuse to form aligned myotubes, e.g., within a bioreactor or other

Certain structures and methods described herein can be useful, for example, by providing meat and other animal derived products for human consumption. Certain embodiments of structures and methods described herein may offer certain advantages as compared to existing agriculture-based methods of meat production, for example, by significantly reducing the number of animals bred for slaughter, thus decreasing the number of foodborne illnesses, diet related diseases, and the incidence of antibiotic resistance and infectious disease (e.g., zoonotic diseases such as Nipah virus and influenza A). In some cases, reducing the number of livestock worldwide may also have an effect on the environmental risks associated with agricultural farming due to, for example, ammonia emissions which contribute significantly to acid rain and acidification of ecosystems. In addition, in some instances, livestock, such as pigs and cows are a major agricultural source of greenhouse gases worldwide. In some embodiments, the structures and methods described herein may allow meat and other animal-derived products to be produced or cultivated in vitro, e.g., using blood and tissue donations obtained from living livestock donors (e.g., not intended for slaughter for human consumption). As a non-limiting example, certain embodiments as described herein are generally directed to a product comprising a muscle replica, a fat replica, and a lysate of red blood cell.

The above discussion is a non-limiting example of one aspect generally directed to microcarriers or other scaffolds comprising fibrin. These can be used for growing cells such as myoblasts, e.g., to produce myotubes in a cultivated meat product, or another cell-based animal-derived product. However, other embodiments are also possible besides those discussed above. Accordingly, more generally, various aspects are directed to various systems and methods for producing cultivated meat and other cell-based animal-derived products, as discussed herein.

For example, certain aspects are generally directed to cultivated animal-derived products, such as cultivated meat, or other products. These may be produced, for example, using cells taken from an animal, but then the cells are cultured in vitro, e.g., using bioreactors, flasks, petri dishes, microwell plates, or other cell culture systems. Many cell culture systems will be known to those of ordinary skill in the art. This is in stark contrast to traditional techniques of sacrificing animals and harvesting their meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro [symbol] than naturally as part a living animal.

A variety of products may be formed from cells cultured in vitro. For instance, in certain embodiments, the products may form “cultivated meat,” or meat that is intended to be eaten, for example, by humans. It will be appreciated that, because it is to be eaten, such products will often be formed of edible or digestible materials, e.g., materials that can be digested, or degraded to form generally nontoxic materials within the digestive system. For instance, the cultivated meat may contain animal-derived cells (e.g., derived from a chicken, a cow, a pig, a sheep, a goat, a deer, a fish, a duck, a turkey, a shrimp, or other animals that are commonly recognized for widespread human consumption), such as muscle cells, fat cells, or the like. The cells may be wild-type or naturally-occurring cells (e.g., harvested from an animal), although in some embodiments, the cells may include genetically engineered cells, e.g., engineered in a way to increase proliferation. In addition, in some embodiments, the cultivated meat product may contain other edible materials, such as plant-originated materials. Non-limiting examples of edible materials include proteins, carbohydrates, sugars, saccharides, plant-based fats, etc., as well as polymers formed from these (for example, polylactic acid, polyglycolic acid, cellulose, etc.). In some cases, the edible materials may be digested to form nutrients, e.g., such as amino acids, sugars, etc. that have nutritional value, for example, when taken up into the body. However, in some cases, the edible materials cannot be digested, and/or can be digested to form non-nutrients that cannot be absorbed as nutrients, but can be passed through the digestive system without detrimental effects.

In addition, it should be understood that the invention is not limited to only cultivated meat products. In some cases, products such as those described herein may be cultivated from animal-derived cells, but the product is not necessarily one that is intended to be eaten. For instance, cells from an animal may be cultured to form various organs that can be harvested, such as skin, hair, fur, or the like. Thus, as a non-limiting example, leather, cultivated fur, etc. can be formed by growing cells in culture, for example as discussed herein, without the traditional method of sacrificing animals to harvest their skin or other organs.

In certain embodiments, the cultivated meat products may be grown on microcarriers or other types of scaffolds, which may comprise fibrin in some embodiments. For example, cells derived from an animal may be seeded onto microcarriers or scaffolds, and grown in vitro, e.g., in a bioreactor or other cell culture systems such as are described herein, to produce a cultivated meat product (or other cultivated animal-derived product). In some [symbol] product thus formed can be used without additional processing. As a non-limiting example, a cultivated meat product may be grown by seeding myoblasts on microcarriers or scaffolds, then growing them within a bioreactor to form a muscle replica or a cultivated meat product, etc. The cultivated meat product may not require subsequent separation or processing steps to convert the cultured cells into a product ready to be cooked or otherwise be used, e.g., as meat. However, it should be understood that in other embodiments, additional steps may be used to convert the muscle replica grown within the bioreactor into a cultivated meat product, or other cultivated animal-derived product.

As mentioned, the cells seeded on the microcarriers or other types of scaffolds may arise or be derived from any suitable animal. Non-limiting examples of animals typically consumed as food include chicken, cow, pig, sheep, goat, deer, fish, duck, turkey, shrimp, or any other suitable animals. In addition, in some cases, the cells may be cells that are not from an animal intended to be consumed by humans as food. For instance, as discussed herein, in some cases, the cells may be cultured to grow leather or cultivated fur, and may be derived from an appropriate animal type, e.g., mink or racoon. In yet another embodiment, the cells may be cultured to grow a product that is to be implanted in a subject. For instance, the cells may be derived from a human, and the product may be a muscle or other organ to be implanted in a human. In one set of embodiments, the cells are derived from the subject (e.g., a human subject) that will receive the implant; this may be useful, for example, to avoid an immunological reaction with the implanted product. As another non-limiting example, organs, tissues, etc. of endangered animals can be grown in accordance with certain embodiments, for example, tiger liver, rhinoceros horn, etc.

In addition, in some embodiments, other types of cells may be seeded on a microcarrier or other type of scaffolds, e.g., in addition (or instead of) myoblasts. In some embodiments, other cell types may comprise fibroblasts, epithelial cells, lymphocytes, and macrophages. In other embodiments, the other cell types may comprise a skin cell, a blood cell, a fat cell, a nerve cell, a sex cell, a stem cell, or other cell types. As a non-limiting example, adipose cells can be added to produce fat within the final cultivated meat product. Additional non-limiting examples include mesenchymal stem cells, bone marrow-derived stem cells, embryonic stem cells, induced pluripotent stem cells, adipose-derived stem cells, etc. In some embodiments, stem cells may be triggered to differentiate into a more specialized cell type. For example, in one embodiment, pluripotent stem cells may be stimulated to differentiate into neural progenitor cells, epithelial stem cells, cardiac progenitor cells, hematopoietic stem cells intestinal stem cells, lung cells, hepatocyte [symbol] cells, pancreatic progenitor cells, etc. In another non-limiting example, mesenchymal stem cells can be differentiated into white fat cells, brown fat cells, skeletal muscle, smooth muscle cells, etc. Cells such as these may be derived from the same animal species as the myoblasts, and/or the cells may be derived from different animal species. Accordingly, one or more than one type of cell may be seeded, e.g., sequentially and/or simultaneously, etc.

Accordingly, in one set of embodiments, cells are seeded onto scaffolds. A scaffold may define a substrate that the cells are able to divide and proliferate on, e.g., forming tissue that forms the basis of the cultivated meat product. A variety of cell scaffold structures can be used, including scaffolds known by those of ordinary skill in the art. The scaffold may thus have any suitable size or shape. In some cases, the scaffold may be anisotropic, i.e., not exhibiting radial or spherical symmetry. In addition, in certain embodiments, the scaffold may be relatively solid, or have holes or pores. Thus, for example, the scaffold may have any suitable degree of porosity. One or more than one scaffold may be present. For instance, in certain cases, the scaffold comprises one or a plurality of microcarriers, e.g., as described herein. If more than one scaffold is present, the scaffolds may be independently the same or different. In addition, in certain embodiments, the scaffold may have one or more grooves, e.g., as discussed herein.

In one set of embodiments, a scaffold may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, or less than 1 mm. In addition, in some cases, the microcarriers may have a maximum internal dimension that is at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. Combinations of any of these dimensions are also possible in some embodiments.

The scaffold may comprise any suitable material. For example, in one set of embodiments, the scaffold may comprise fibrin, or another edible material. This may be useful for applications such as cultivated meat, where the cultivated animal-derived product will be eaten, e.g., by humans or other animals. In some embodiments, the microcarriers may comprise a hydrogel, e.g., a fibrin hydrogel, or other hydrogels such as those described herein.

In some cases, at least 50 wt %, at least 60 wt %, at least 70 wt %, at least 80 wt %, at least 90 wt %, or substantially all of a scaffold is formed from fibrin, and/or another edible [symbol] the fibrin may arise from any suitable source. For example, the fibrin may arise from a non-human animal, such as a non-human mammal. Non-limiting examples include cows, pigs, sheep, goats, or the like. In some cases, the fibrin may arise from the blood of such an animal. For instance, in some embodiments, the fibrin may be prepared by acquiring blood or blood plasma from an animal, and processing it to produce fibrin. In some embodiments, plasma may be prepared by plasma apheresis of living animal periodically without slaughtering animal. For example, apheresis may be performed at least 1 time per month, at least 2 times per month, at least 3 times per month, and at least 4 times per month. In another example fibrin may be prepared from animal blood produced in slaughterhouses. For example, in one set of embodiments, the blood is exposed to a protease inhibitor such as thrombin, which may cause fibrinogen to clot to form fibrin. The fibrin may be harvested, and used as discussed herein, e.g., to produce scaffolds such as microcarriers. In addition, in some cases, fibrin may be obtained from fibrinogen, which may be bought commercially, obtained from blood plasma, or the like.

In some cases, the blood may be acquired from the animal without killing the animal. For instance, blood may be withdrawn from the animal at spaced intervals, so as to allow the animal time to recover and produce new blood. For instance, blood may be withdrawn from the animal every 4 weeks, every 6 weeks, every 2 months, or the like. Additional details may be found in a patent application entitled “Methods and Systems of Preparing Cultivated Meat from Blood or Cellular Biomass,” filed on Nov. 15, 2021, U.S. Pat. Apl. Ser. No. 63/279,631, incorporated herein by reference in its entirety.

The fibrin may be processed to form a scaffold. In one set of embodiments, the scaffold may take the form of one or more microcarriers. The microcarriers may have any shape or size. In some cases, more than one type of microcarrier may be present, e.g., some of which may have various materials, shapes, sizes, etc., such as are described herein. For example, in some embodiments, at least some of the microcarriers may be substantially spherical or exhibit spherical symmetry, although in other embodiments, at least some of the microcarriers may be non-spherically symmetric (for example, triangular) or may be anisotropic. In addition, in certain cases, at least some of the microcarriers may have a plurality of grooves, e.g., as discussed herein.

In certain embodiments, the microcarriers may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, less than 1 mm, less than 0.5 mm, less than 0.3 mm, [symbol] mm, less than 0.1 mm, less than 0.05 mm, less than 0.03 mm, less than 0.02 mm, or less than 0.01 mm. In addition, in some cases, the microcarriers may have a maximum internal dimension that is at least 0.01 mm, at least 0.02 mm, at least 0.03 mm, at least 0.05 mm, at least 0.1 mm, at least 0.2 mm, at least 0.3 mm, at least 0.5 mm, at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. In addition, in certain cases, combinations of any of these dimensions are also possible. As non-limiting examples, the microcarriers may have a maximum internal dimension of between 10 mm and 30 mm, between 5 mm and 20 mm, between 3 mm and 10 mm, between 50 mm and 70 mm, between 1 mm and 3 mm, etc. In some cases, the maximum internal dimension is the length of longest straight line that can be contained entirely within the microcarrier and/or the interior of the microcarrier (e.g., if the microcarrier defines a hollow sphere).

In some cases, however, some or all of the microcarriers may not necessarily be spherical. For example, at least some of the microcarriers may have shapes such as cubical, rectangular solid, triangular, tetrahedral, octahedral, irregular, etc. In some cases, at least some of the microcarriers have a shape that is substantially planar. For instance, the microcarrier may have a generally rectangular shape where the smallest dimension of the rectangular solid is substantially smaller than either of the other two dimensions, for example, by a factor of at least 3, at least 5, or at least 10, etc.

In addition, in certain cases, at least some of the microcarriers have a relatively large surface to volume ratio. This may be important, for example, in embodiments where the microcarriers contain a plurality of grooves, e.g., as discussed herein. In contrast, a perfect sphere would have the smallest possible surface to volume ratio for a given volume of material. As a nonlimiting example, the surface to volume ratio may be at least 100, at least 200, at least 300, etc., e.g., for a sheet thickness of 0.01 mm surface and an area of 1 mm×10 mm.

Fibrin itself may be edible. The microcarrier or scaffold may comprise, in addition to or instead of fibrin, other edible materials in certain embodiments. In addition, it should be understood that the scaffold is not limited to only edible or degradable materials. In other embodiments, the scaffold may comprise materials, such as polymers, that are not necessarily edible and/or degradable. Non-limiting examples of such materials include natural polymers such as proteins (e.g., silk, collagen, gelatin, fibrinogen, elastin, keratin, actin, myosin, etc.), polysaccharides (e.g., cellulose, amylose, dextran, chitin, glycosaminoglycans), or the like. [symbol] holes include polymers such as polylactic acid, polyglycolic acid, poly(lactic-co-glycolic acid), polyhydroxyalkanoates, polycaprolactones, etc., bioactive ceramics such as hydroxyapatite, tricalcium phosphate, silicates, phosphate glasses, glass-ceramic composites (such as apatite-wollastonite), etc., or the like.

While almost anything can physically be eaten, materials that are edible include those that are found naturally occurring in foods that are commonly eaten by significant percentages of the general population. Examples of edible materials include, but are not limited to proteins or peptides, polysaccharides, carbohydrates, or the like. In some cases, such materials may be broken down by the digestive system to produce nutrients such as amino acids, monosaccharides, simple sugars, etc. However, in some cases, the edible materials need not be digestible into such nutrients. Specific non-limiting examples of edible materials include cellulose, chitin, collagen, soy protein, mycelium, gelatin, alginate, etc.

Additionally, in some but not all embodiments, the scaffold may comprise a plant-originated material, such as a plant-originated protein. Such plant-originated materials may be harvested directly from a plant, be grown in vitro (e.g., in cell culture from a culture initially originating in a plant), be synthetically produced (e.g., without using a plant, e.g., chemically produced), etc. Examples of protein-originated material include, but are not limited to, cellulose or certain proteins, such as prolamin, zein, fibrin, gliadin, hordein, secalin, kafirin, avenin, gliadine, 2S albumin, globulin, glutelin, etc. The plant that material originates from may be any plant, including but not limited to food crop plants. Non-limiting examples of plants include, but are not limited to, wheat, barley, rye, corn, sorghum, oats, quinoa, hemp, potato, soy, etc. Additional examples of such materials include those described in U.S. Ser. No. 63/159,403, filed Mar. 10, 2021, entitled “Constructs for Meat Cultivation and Other Applications,” by Khademhosseini, et al., incorporated herein by reference in its entirety.

The microcarrier, or other scaffold may also be biocompatible in some instances. In addition, in certain embodiments, the scaffold may comprise a polymer, e.g., one that is biodegradable. For example, in some cases, the scaffold may be one that begins to spontaneously degrade (for example, via hydrolysis reactions, dissolution, etc.) when maintained in contact with water, e.g., for at least 12 hours.

In one aspect, at least some of the microcarriers or other types of scaffolds may have one or more grooves defined therein. (However, it should be understood that such grooves are not always required in other embodiments.) Examples of such grooves include those discussed in U.S. Ser. No. 63/159,403, filed Mar. 10, 2021, entitled “Constructs for Meat [symbol] and Other Applications,” by Khademhosseini, et al., incorporated herein by reference in its entirety. Without wishing to be bound by any theory, it is believed that grooves may promote cellular alignment during growth on the microcarriers or other types of scaffolds. Thus, for example, myoblasts seeded within grooves on microcarriers may be induced to grow together to form substantially aligned myotubes, e.g., that are substantially parallel to each other. This can result in muscle fibers can be grown on the microcarriers. As this is a biological system, those of ordinary skill in the art would understand that the myotubes will not necessarily grow to be perfectly parallel to a high degree of mathematical precision. Nonetheless, the myotubes may still be readily identified as having substantially parallel myotubes within the cultivated animal-derived product, for example, as opposed to myotubes grown on spherical particles not containing grooves, where the myotubes are formed randomly from the myoblasts. For instance, the myotubes may exhibit a strong preference to the direction of the grooves, e.g., having an average directionality that varies by less than 20°, less than 15°, less than 10°, or less than 5° relative to the direction of the grooves.

Thus, in some embodiments, the grooves may be positioned or sized within the microcarriers or other scaffolds to allow the myoblasts to be directional or aligned, e.g., to allow them to fuse together to become myotubes. One or more grooves may be present. For instance, a microcarrier or other scaffold may have at least 2, at least 3, at least 4, at least 5, at least 7, at least 10, at least 12, at least 15, at least 20, at least 25, at least 30, at least 40, at least 50, at least 70, or at least 100 or more grooves defined therein. If more than one type of microcarrier or scaffold is present, they may independently have the same or different numbers of grooves. In some cases, the average number of grooves present within the microcarriers may have the ranges described here. The grooves may be positioned in any orientation on the microcarriers. For instance, the grooves may be substantially parallel to each other, e.g., to promote the formation of substantially aligned myotubes. The grooves may also have any profile, e.g., square or rectangular, and any aspect ratio (i.e., width to height).

The microcarriers, or other scaffolds such as described herein, may be formed using any suitable technique, according to certain aspects. Non-limiting examples include extrusion, electrospinning, 3D-printing, molding, injection molding, or the like, e.g., of a precursor solution or a hydrogel block, etc. For example, a microcarrier or other scaffold maybe formed by milling, chopping, homogenizing, or otherwise processing hydrogel blocks. As still another non-limiting example, fibrin hydrogel blocks can be formed into millimeter- [symbol] carriers using high speed homogenizers, or the like. In yet another other non-limiting example, cells may be confined on an engineered surface or material having a micro-nano-topography as contact guidance, or by applying mechanical forces generated either by the contractile activity of the cells or by an external strain.

In one set of embodiments, materials that will be used to form a microcarrier (e.g., comprising fibrin, etc.) may be formed into a paste or other mixture that is extruded, e.g., at low temperatures (e.g., temperatures below 20° C., 15° C., 10° C., or 5° C., etc.) and/or into a water bath to solidify and/or coagulate the materials into microcarriers. The mixture may have, for example, at least 20%, at least 30%, at least 40%, at least 50%, at least 60%, or at least 70% material (e.g., fibrin), and/or no more than 80%, no more than 70%, no more than 60%, no more than 50%, no more than 40%, or no more than 30% material, by weight. In some cases, combinations of any of these ranges are also possible, e.g., the mixture may have between 40% and 60% material (e.g., fibrin), between 20% and 80% material, between 30% and 50% material, etc. In some cases, the materials may be dissolved and/or suspended in a suitable liquid, e.g., water, a strong alcohol (e.g., 70% to 80% aqueous solution by volume), an acid solution, an alkaline solution, or the like. These percents are percent by weight.

In some embodiments, the microcarriers (or other scaffolds) may be formed to have any of a wide variety of shapes, such as flakes, plates, fibers, whiskers, or the like, e.g., having dimensions such as any of those described herein. In addition, as previously noted, in certain embodiments, some of these shapes may contain grooves.

As a non-limiting example, the microcarriers may have the form of fibers, e.g., having an average length of at least 1 micrometer, at least 2 micrometers, at least 3 micrometers, at least 4 micrometers, at least 5 micrometers, at least 10 micrometers, at least 20 micrometers, at least 30 micrometers, at least 40 micrometers, at least 50 micrometers, at least 100 micrometers, at least 200 micrometers, at least 300 micrometers, at least 400 micrometers, at least 500 micrometers, at least 1 mm, at least 2 mm, at least 3 mm, at least 4 mm, at least 5 mm, at least 1 cm, at least 2 cm, at least 5 cm, at least 10 cm, at least 20 cm, at least 30 cm, at least 50 cm, etc. In addition, in certain embodiments, the fibers may have an average length of no more than 100 cm, no more than 50 cm, no more than 30 cm, no more than 20 cm, no more than 10 cm, no more than 5 cm, no more than 4 cm, no more than 3 cm, no more than 2 cm, no more than 1 cm, no more than 5 mm, no more than 4 mm, no more than 3 mm, no more than 2 mm, no more than 1 mm, no more than 500 micrometers, no more than 400 micrometers, no more than 300 micrometers, no more than 200 micrometers, no more than 100 micrometers, etc. Combinations of any of these are also possible, e.g., the fibers may [symbol] average length of between 200 micrometers and 500 micrometers, between 500 micrometers and 5 mm, between 300 micrometers and 1 mm, between 10 micrometers and 400 micrometers, etc.

In some cases, the microcarriers (or other scaffolds) may be purified, e.g., by extracting impurities prior to use, e.g., prior to seeding with cells. For example, impurities such as citric acid or ethanol may interfere with cell culture, and/or may interfere with the taste of the cultivated meat product. Thus, in some cases, such microcarriers or other scaffolds may be exposed to water, e.g., washed, to remove potential contaminants.

In addition, in certain embodiments, the microcarriers or other scaffolds may be sterilized before use, e.g., prior to seeding with cells. A variety of techniques for sterilizing the microcarriers can be used, including but not limited to, applying ultraviolet light, gamma radiation, or high temperatures (e.g., a temperature of at least 100° C.) to the microcarriers. Those of ordinary skill in the art will be aware of various sterilization techniques that may be used.

In some embodiments, the microcarriers may be formed into a cultivated meat product, or other cultivated animal-derived product. For example, cells such as non-human animal cells may be grown on microcarriers or scaffolds, e.g., as discussed herein. Non-limiting examples include muscle cells (e.g., myoblasts), adipose (fat) cells, or the like, and the cells may arise from the same or different species. In some cases, relatively large quantities of product may be prepared, e.g., by growing the cells in a bioreactor or other in vitro cell culture system, until at least a certain size or mass is reached. For example, the cells may be grown until they form a product that is, for example, at least 10 g, at least 25 g, at least 50 g, at least 100 g, at least 300 g, at least 1 kg, etc. Those of ordinary skill in the art will be aware of bioreactors and other cell culture systems.

In addition, in some cases, other materials may be added to the cultivated meat product, or other cultivated animal-derived product. As a non-limiting example, in some cases, a fat replica may be added to the product. One example of a fat replica is a fat replica comprising a fat emulsion and a hydrogel. A variety of fat replicas, including this, are discussed in a patent application entitled “Systems and Methods of Producing Fat Tissue for Cell-Based Meat Products,” filed on Nov. 15, 2021, U.S. Pat. Apl. Ser. No. 63/279,642, incorporated herein by reference in its entirety. Another non-limiting example are hemes. Examples of such hemes are described in a patent application entitled “Production of Heme for Cell-Based Meat Products,” filed on Nov. 15, 2021, U.S. Pat. Apl. Ser. No. 63/279,644, incorporated herein by reference in its entirety. One or more of these may be present within a product, such as a cultivated meat product, in accordance with various embodiments such as discussed herein.

For example, in some embodiments, the cells may be mixed with a fat replica, e.g., within the cultivated meat product, or other cultivated animal-derived product. In some embodiments, the fat replica comprises an emulsion. In some cases, the emulsion comprises a fat emulsion, and a crosslinked hydrogel. In some embodiments, the fat comprises non-human animal fat. The fat may also comprise a plant-derived fat. Non-limiting examples of plant-derived fats include vegetable oil, sunflower seed oil, and corn oil. In yet other embodiments, the fat is saturated fat, unsaturated fat, or both.

In addition, in certain embodiments, a surfactant may be present, and may be used to disperse the fat and/or stabilize the emulsion. Non-limiting examples of surfactants include phospholipids, monoglyercols, diglycerols, propylene glycol monoesters, lactylate esters, polyglycerol esters, sorbitan esters, ethoxylated esters, succinate esters, fruit acid esters, acetylated monoglycerols, acetylated diglycerols, phosphate monoglycerols, phosphate diglycerols, sucrose esters, etc. Those of ordinary skill in the art will be familiar with surfactants, including ones that may be edible.

In some embodiments, the fat replica may comprise a hydrogel, which may be crosslinked in some cases. In some cases, the hydrogel comprises non-human blood plasma. For example, the non-human blood plasma may contain fibrin, which may be crosslinked to form a hydrogel. Other non-limiting examples of hydrogels that can be used within the fat replica include proteins (for example, collagen, gelatin, etc.), polymers (for example, polylactic acid, polyglycolic acid, etc.), carbohydrates (for example, alginate, hyaluronan, chitosan, cellulose, hydroxymethyl cellulose, etc.), or the like.

The hydrogels can be non-covalently and/or covalently crosslinked. Non-covalent hydrogels may be stabilized in some embodiments by hydrogen bonding, van der Waals interactions (e.g., hydrophobic interactions), etc. Covalent hydrogels may be formed, for example, by adding a crosslinking agent, bearing a first coupling group, to a crosslinkable material, bearing a second coupling group. The coupling groups can be any functional groups known to those of skill in the art that together form a covalent bond, for example, under mild reaction conditions or physiological conditions. Examples of coupling groups include, but are not limited to, maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl [symbol] azides, diazirines, benzophenone, epoxides, carbonates, anhydrides, sulfonyl chlorides, cyclooctynes, aldehydes, and sulfhydryl groups, etc. In some embodiments, coupling groups may include free amines (—NH₂), free sulfhydryl groups (—SH), free hydroxide groups (—OH), carboxylates, hydrazides, alkoxyamines, etc. In some embodiments, a coupling group can be a functional group that is reactive toward sulfhydryl groups, such as maleimide, pyridyl disulfide, or a haloacetyl.

In some embodiments, the crosslinking agent and crosslinkable material are functionalized with groups used in “click” chemistry. Examples of “click” chemistry groups include a 1,3-dipole, such as an azide, a nitrile oxide, a nitrone, an isocyanide, etc., which link with an alkene or an alkyne dipolarophile, or the like. Exemplary dipolarophiles include any strained cycloalkenes and cycloalkynes, including, but not limited to, cyclooctynes, dibenzocyclooctynes, monofluorinated cyclcooctynes, difluorinated cyclooctynes, and biarylazacyclooctynone.

In yet another embodiment, a product such as a cultivated meat product may be mixed with the lysate of non-human red blood cells to impart the cultivated meat product with the red appearance of native red muscle. In some cases, at least 1%, at least 3%, at least 5%, at least 10%, at least 20%, of the product comprises the lysate of non-human red blood cells. In some embodiments, the non-human red blood cells are lysed within 24 hours of withdrawal from a non-human living donor. See, e.g., a patent application entitled “Methods and Systems of Preparing Cultivated Meat from Blood or Cellular Biomass,” filed on Nov. 15, 2021, U.S. Pat. Apl. Ser. No. 63/279,631, incorporated herein by reference.

As mentioned, in some aspects, a product such as a cultivated meat product may be produced within a bioreactor or other cell culture system. A wide variety of bioreactors can be used in various embodiments including, but not limited to, suspension bioreactors, continuous stirred-tank bioreactors, rocker bioreactors, airlift bioreactors, fixed bed bioreactors, bubble column bioreactors, fluidized bed bioreactors, packed bed bioreactors, or the like.

In some cases, cells may be seeded on microcarriers or other scaffolds, then introduced into the bioreactor or other cell culture system. Those of ordinary skill in the art will be familiar with techniques for seeding cells on a scaffold. For instance, the scaffold may be exposed to a suspension containing animal-derived cells, which are allowed to settle from the suspension onto the scaffold. In some cases, one or more than one type of cell may be present in suspension and allowed to settle.

In some cases, a product can be formed within the bioreactor without additional [symbol] for example, without separating the cells or tissues grown within the bioreactor. However, in other cases, some separation and/or processing of the cells may be used. As a non-limiting example, myotubes may be grown within a bioreactor or other cell culture system such as those described herein to produce a muscle replica. In some embodiments, such muscle replicas may be processed, e.g., by adding a fat replica to produce a cultivated meat product having any desired ratio of muscle to fat in it. For instance, the ratio of muscle to fat may be at least 95:1, at least 90:1, at least 70:1, at least 50:1, at least 30:1, at least 20:1, at least 10:1, at least 5:1, at least 1:1, etc. by weight. One non-limiting example of a fat replica is a fat replica comprising a fat emulsion and a hydrogel, e.g., as discussed in a patent application entitled “Systems and Methods of Producing Fat Tissue for Cell-Based Meat Products,” filed on Nov. 15, 2021, U.S. Pat. Apl. Ser. No. 63/279,642, incorporated herein by reference in its entirety.

A variety of techniques may be used to grow cells within the bioreactor or other cell culture system. For instance, the cells may be grown at body temperature (e.g., about 38.5° C. for cow cells, about 41° C. for chicken cells, about 39-40° C. for pig cells, about 40-42° C. for duck cells, etc.). In some embodiments, during cultivation, the cells may have a shear stress applied to them of at least 0.005 newton/meter squared, of at least 0.1 newton/meter squared, of at least 0.2 newton/meter squared, of at least 0.3 newton/meter squared, of at least 0.4 newton/meter squared, of at least 0.5 newton/meter squared, of at least 0.6 newton/meter squared, of at least 0.7 newton/meter squared, of at least 0.8 newton/meter squared, etc.

In some embodiments, cells within the bioreactor or other cell culture system may be induced to differentiate, e.g., by adding suitable factors and/or altering the cell culture conditions therein. As a non-limiting example, myoblasts may be grown in serum, while removing or reducing the serum from the myoblasts may cause the myoblasts to differentiate to from myotubes. For instance, in one set of embodiments, the serum may be reduced from 10% to 2% to induce differentiation of myoblasts. Those of ordinary skill in the art will be aware of methods and systems to induce differentiation in cells.

In some embodiments, the serum can be obtained from commercial vendors. In certain cases, serum may be obtained from fresh whole blood. As a non-limiting example, the blood may be drawn within 24 hours from a living non-human animal donor, e.g., one that is not being slaughtered for meat. See, for example, “Methods and Systems of Preparing Cultivated Meat from Blood or Cellular Biomass,” filed on Nov. 15, 2021, U.S. Pat. Apl. Ser. No. 63/279,631, incorporated herein by reference in its entirety.

In one embodiment, the cell-based meat product may be grown in a bioreactor, or [symbol] cell culture system, comprising a cell growth medium. In one embodiment, the cell growth medium comprises an animal derived product, for example, platelet rich plasma (PRP), platelet poor plasma, platelet lysate (PL), platelet concentrate, a lysate of red blood cells, optionally comprising other nutrients, or the like. The cell growth medium may be used for the production of cell-based meat and/or to enhance the proliferation of primary cells, stem cells such as myoblasts, fibroblasts, adipocyte, vascular, osteoblasts, tenocyte, neural cells, etc. These cells may be isolated from human or non-human animals, grown in vitro, etc. These may include but are not limited to humans, cows, sheep, swine, horses, goats, camels, whales, fishes, crabs, shrimp and the like. In some embodiments, the blood products may be obtained from the blood of animals destined to slaughtered for food.

In one embodiment, the platelet rich plasma (PRP) may be derived from whole blood from which red blood cells and white blood cells have been removed, such as by centrifugation, filtration, or other techniques known to those of ordinary skill in the art. Platelet rich plasma (PRP) may be generally categorized based on its leukocyte and fibrin content as (1) leukocyte-rich PRP (L-PRP), (2) leukocyte reduced PRP (P-PRP); (3) leukocyte reduced/pure PRP, or (4) leukocyte platelet-rich fibrin/pure platelet-rich fibrin (L-PRF). The platelet-rich plasma may be a blood derived composition having an increased concentration of platelets, compared to normal blood. For example, the PRP may have at least double, at least five times, or at least ten times or more the normal concentration of platelets in blood. In addition, in accordance with another embodiment, the platelet rich plasma may contain a variety of endogenous growth factors, such as transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, etc.

In one embodiment, the platelet concentrate (PC) may be derived from the platelet rich plasma (PRP), for example, by centrifugation. In some embodiments, the concentration may be at least 10³ platelets/mL, at least 10⁴ platelets/mL, at least 10⁵ platelets/mL, at least 10⁶ platelets/mL, at least 10⁷ platelets/mL, at least 10⁸ platelets/mL, at least 10⁹ platelets/mL, at least 10¹⁰ platelets/mL, etc. In some embodiments, donated platelet concentrates may be stored at 4° C. prior to use for at least 1 day, at least 2 days, at least 3 days, at least 4 days, at least 5 days, at least 6 days, or at least 7 days after donation. In another embodiment, expired human platelet concentrate may be obtained from blood banks, hospitals, and other institutions that routinely collect and store platelet rich plasma, and used as an additive in the cell growth medium.

In one embodiment, the platelet concentrate may impart the cell growth medium with antimicrobial properties. Platelets have certain properties similar to immune cells, and can in some cases induce potent anti-inflammatory responses when exposed to a number of chemical and biological triggers, for example, liposaccharide protein (LPS). In some embodiments, the platelet concentrate may be added to the cell culture medium and stimulated by treatment with platelet activating reagents, for example, calcium, thrombin, citrate, EDTA, plasminogen, and other platelet activating reagents known to those skilled in the art, e.g., to release antimicrobial molecules that may neutralize common bacterial, fungal, or viral food pathogens. In some embodiments, an acellular antimicrobial cell growth medium may be prepared by first culturing the platelet concentrate in the cell culture medium, stimulating them to release their antimicrobial payload, and then separating the antimicrobial cell growth medium from the platelet concentrate.

In one embodiment, the platelet concentrate may be lysed, for example by freeze-thawing or physical shearing (e.g. sonication or homogenization, etc.), to yield a platelet lysate (PL) comprising a plurality of cytokines and growth factors (e.g. transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, etc.) that in some embodiments may enhance cell proliferation, for example, of myoblasts and adipocytes. In some embodiments, the platelet lysate comprises human platelets, and/or non-human platelets. For example, in one embodiment, the platelet rich plasma may include bovine platelet rich plasma.

In one embodiment, the cell growth medium comprises a combination of a platelet lysate (PL) and a platelet rich plasma (PRP). In one embodiment, the PL/PRP comprise at least 2 to 20% w/v, at least 5-15% w/v, or at least 10% w/v of the cell culture growth medium. In another embodiment, the total platelet component in the cell growth medium is at least 2 to 5 mg/mL, at least 2 to 10 mg/mL, at least 2 to 20 mg/mL, or at least 9 to 11 mg/mL.

The cells may be grown within the bioreactor or other cell culture system for any suitable length of time, e.g., to produce a cultivated product. For example, the cells may be grown for at least 3 days, at least 5 days, at least 1 week, at least 2 weeks, at least 3 weeks, at least 4 weeks, etc.

In some cases, the scaffold or microcarrier containing cells may be usable with the scaffold or microcarrier in place. For example, the cells and the scaffold or microcarrier [symbol] form a cultivated meat product (for example, if the scaffold or microcarrier is edible and/or degradable), or other cultivated animal-derived product. In some embodiments, the non-human cell-to-microcarrier ratio in the product is at least 95:5, at least 85:15, at least 75:25, at least 25:75, at least 15:85, or at least 5:95. However, in some embodiments, the cells may be separated from the scaffold or microcarrier. For example, the scaffold or microcarrier may be removed and reused or discarded, while the cells may be used without the scaffold present. Thus, for example, if the cells are used in a cultivated meat product or other cultivated animal-derived product, the scaffold or microcarrier may not necessarily be edible and/or degradable.

In some embodiments, a cultivated meat product may be formed by mixing a muscle replica, a fat replica (e.g., comprising a fat emulsion and a hydrogel), and a lysate of non-human red blood cells. In some embodiments, the non-human cell to fibrin microcarrier ratio is at least 95:5, at least 85:15, at least 75:25, at least 25:75, at least 15:85, at least 5:95, etc. In certain other embodiments, the percent by weight of muscle replica to fat replica is at least 5:95, at least 10:90, at least 15:85, at least 20:80, at least 30:70, etc.

In some instances, a product such as a cultivated meat product further comprises binding agents that hold the various components together. Exemplary embodiments include transglutaminase, non-human plasma, fibrinogen, soy isolate, a soy concentrate, a soy milk, an egg, a soy flour, a wheat gluten isolate, or a pea isolate.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; U.S. Provisional Patent Application Ser. No. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived [symbol] U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.”

The following examples are intended to illustrate certain embodiments of the present disclosure, but do not exemplify the full scope of the disclosure.

### Example 1

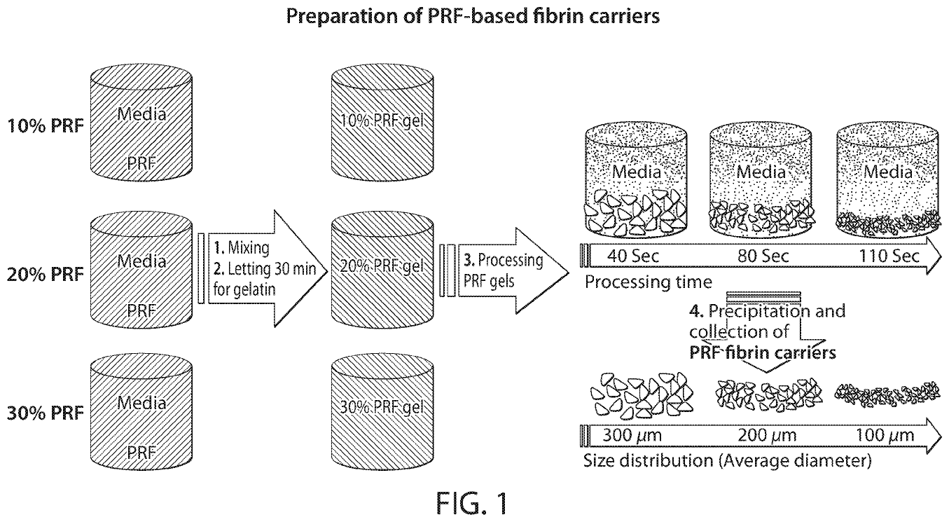

This example illustrates one method for producing fibrin microcarriers by milling preformed fibrin hydrogels (FIG. 1). In one embodiment, fibrin hydrogels are formed using non-human blood plasma isolated from freshly procured whole blood (such as from a chicken, goat, cow, turkey, etc.). Fresh whole blood can be obtained, for example, by placing a catheter into the animal's vein, engaging the syringe tip with the catheter and retracting the plunger to remove blood from the animal, disengaging the syringe from the catheter, engaging the syringe tip with a vacutainer and depressing the syringe plunger to transfer the blood into the vacutainer, which contains an anticoagulant. Non-human blood plasma can be obtained from whole blood by centrifuging the whole blood at suitable speeds, such as 1000 g, at 2000 g, at 3000 g, at 4000 g, at 5000 g, etc. After centrifugation, the plasma fraction may be decanted, separating it from the pelleted cell fraction. Plasma rich fibrinogen (PRF), with varying concentrations of fibrinogen, may then be prepared by diluting the non-human blood plasma with cell culture media. In some cases, at least 10%, at least 20%, at least 30%, at least 40%, at least 50%, at least 60%, at least 80%, at least 90% (vol/vol), or substantially all of a PRF is used to form the fibrin microcarriers.

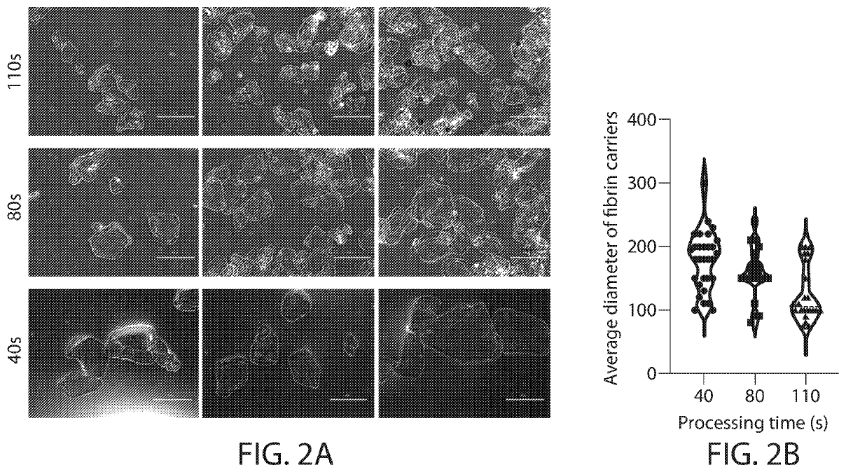

In one embodiment, the PRF was diluted with basal media to a final concentration of 10%, 20%, and 30% (vol/vol), respectively. The solutions were poured into a series of molds at 37° C. for 30 min to allow the fibrinogen time to gel. Here a mold may be a vessel, tank, or any other hollow shape that can hold the solution until gelled (e.g., sheets and rectangular blocks with or without grooves). Once gelled, the fibrin scaffolds were placed into a blender and homogenized between 100 to 1000 rpm for either 40 s, 80 s, or 110 s to yield fibrin microcarriers with flake-like morphologies. In some embodiments, the shape of the microcarrier will depend on the blade geometry and the size of chamber used to homogenize the microcarriers. Fibrin microcarriers were than taken and imaged using optical microscopy and the average diameter of fibrin microcarriers determined using ImageJ by measuring the average diameter (FIG. 2A). The results showed that longer mill times corresponded to smaller microcarriers (FIG. 2B).

### Example 2

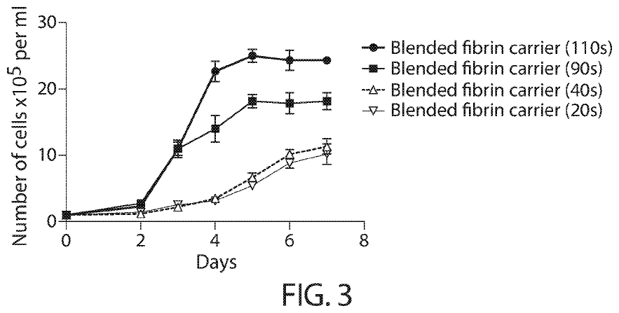

In this example, fibrin microcarriers with a PRF concentration of 20% vol/vol (in balance with DMEM) were prepared as described in Example 1, and milled in a blender for either 20 s, 40 s, 90 s, or 110 s. The fibrin microcarriers (8.8 mg/mL) were placed in bioreactor containing myoblasts (100,000 cells/mL), resuspended in 100 mL of culture media, and mixed for 7 days. The bioreactor was a stir-tank bioreactor, but other reactors including fluidized bed, packed bed, and aerated reactors, etc. may also be used. An aliquot of media was removed once a day and the number of cells per mL was determined. These results indicated that fibrin microcarriers with longer milling times had higher cell densities. It is believed that smaller particles have greater surface to volume ratios, which increases the available surface area for cells to grow and proliferation (FIG. 3).

### Example 3

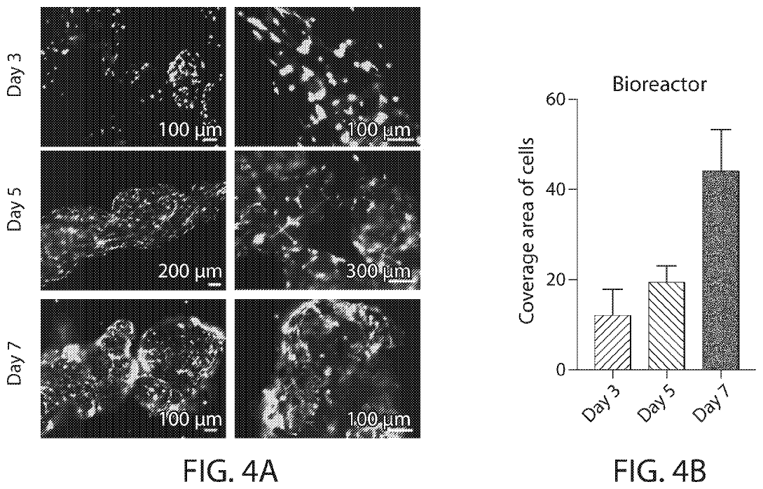

This example tested the ability of fibrin microcarriers to promote growth and proliferation of myoblasts in a bioreactor following encapsulation within a carrier. Fibrin microcarriers were prepared as described in Example 1, except that bovine plasma was suspended in basal media containing bovine myoblasts (100,000 cells/mL) to a final PRF concentration of 10% vol/vol. The mixture was poured into a series of molds and kept 37° C. for 30 min to allow the fibrinogen time to gel. Once gelled, the fibrin scaffolds were placed into a blender and milled for 110 s at 5000 rpm to yield fibrin microcarriers with flake-like morphologies. The fibrin microcarriers were resuspended in growth media comprised of DMEM and 10% platelet rich plasma (PRP), loaded into a bioreactor (total volume, 200 mL), and allowed to cultivate for seven days. On days 3, 5, and 7, sample fibrin microcarriers were removed from the bioreactor, stained with calcein AM, imaged using fluorescence microscopy (See FIG. 4A), and the cell density quantified (See FIG. 4B). These results demonstrate that the cell density increased as a function of culture time, and that cells readily migrated throughout and proliferated within the fibrin microcarriers.

### Example 4

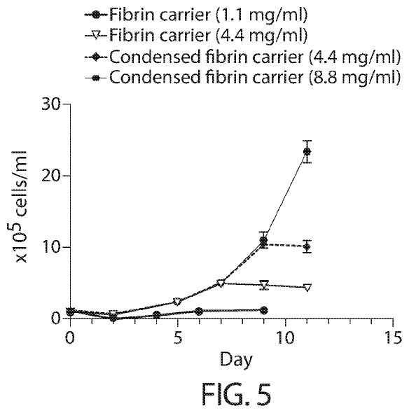

This experiment was designed to determine the effect of temperature and fibrinogen concentration on proliferation kinetics. In yet another embodiment, fibrin microcarriers containing varying concentrations of PRF were prepared by first diluting non-human blood plasma to 1.1 mg/mL, 4.4 mg/L or 8.8 mg/mL with basal media. The various PRF solutions were poured into molds and kept at either 37° C. or 4° C. (referred to as condensed fibrin carriers) for 30 min to allow the fibrinogen time to clot. Once gelled, the fibrin scaffolds [symbol] in a blender for 110 seconds for 5000 rpm and homogenized to yield fibrin microcarriers with flake-like morphologies. The fibrin microcarriers were subsequently placed into a 100 mL bioreactor vessel filled with growth media comprised of DMEM and 10% bovine platelet rich plasma and bovine myoblast cells (final concentration was 100,000 cells/mL) and allowed to culture for 7 days. On days 2, 3, 4, 5, 6, and 7 representative fibrin microcarriers were removed from culture and the cell density quantified by first dissolving the fibrin gel using a trypsin solution (0.25% in EDTA) and the cells imaged and quantified by optical microscopy. These results showed that condensed fibrin microcarriers containing 8.8 mg/mL of PRF exhibited exponential cell growth, compared to uncondensed fibrin microcarriers, which exhibited near-linear proliferation kinetics (FIG. 5).

### Example 5

As mentioned above, in some embodiments, individual microcarriers can be fabricated by techniques such as extrusion, electrospinning, 3D printing, molding a fibrin solution, etc. In this non-limiting example, fibrin microcarriers were formed into fibers using two alternative methods. In one embodiment, solid fibrous fibrin microcarriers were formed by flowing a PRF solution containing a crosslinking agent inside a tubular mold (glass, plastic, or stainless steel), allowing it to gel, and then pushing it out to form solid microtubes.

In yet another embodiment, fibrous fibrin microcarriers were prepared using electrospinning. Electrospinning is generally a fiber production method which uses electric force to draw charged threads of polymer solutions or polymer melts to form fibers. These can have fiber diameters, for example, on the order of hundreds of nanometers. In this example, to produce fibrin fibers via electrospinning, non-human blood plasma (100 vol/vol %) was freeze-dried for 24 h to create a dry non-human blood plasma, which was finely ground using a mortar and pestle before use. The dried non-human blood plasma powder was dissolved in 1,1,1,3,3,3-hexafluoro-2-propanol at a concentration of 200 mg/ml. Once in solution, the non-human blood plasma was loaded into a 3 mL syringe and placed in a syringe pump and the rate set to 2.8 ml/h. A blunt metallic 18-gauge needle was placed on the syringe tip, and the positive voltage lead of a power supply was attached to the needle and set to 25 kV. A grounded piece of aluminum foil was used as a collection plate and was placed 20 cm away from the needle tip. All of these electrospinning processes were performed at 27° C. and 60% humidity.

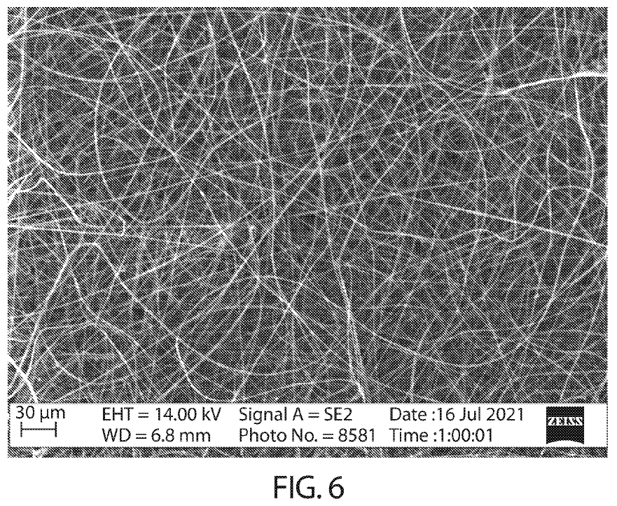

The fiber morphology of the electrospun samples produced in this example was studied via scanning electron microscopy (SEM) operating at 14 kV. Electrospun samples were coated with Au—Pd at a thickness of 100 Angstroms (Å) to reduce charging and produce [symbol] surface (see FIG. 6; scale bar is 30 micrometers). The average fiber diameter of the electrospun fibrin fibers was determined from the SEM images using UTHSCSA ImageTool 3.0 software. Fiber diameter averages and standard deviations were calculated by taking the average of 60 random measurements per micrograph and was determined to be 0.82+/−0.14 micrometers.

### Example 6

This example tested the ability of fibrous fibrin microcarriers generated using electrospinning to promote growth and proliferation of myoblasts in a bioreactor. Electrospun fibrin fibers were created as described in Example 5. 10 mm diameter discs were punched from the electrospun fibrin mats, disinfected (30-minute soak in ethanol, followed by three 10-minute rinses in PBS), and placed in a 48-well plate. The electrospun fibrous fibrin microcarriers were subsequently seeded with 100,000 primary satellite cells (isolated from a cow) in 500 microliters of complete growth media (DMEM low glucose, supplemented with 10% FBS and 1% penicillin/streptomycin, Invitrogen, Carlsbad, CA, USA) and were cultured for up to 10 days in a humidified atmosphere (5% CO₂) at 37° C. The media was changed every other day.

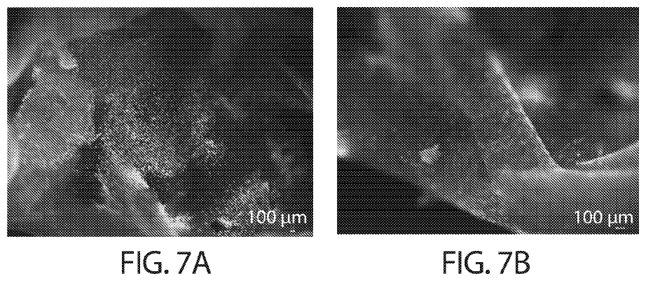

To evaluate cell viability, the electrospun fibrous fibrin microcarriers were stained using a solution containing calcein AM (2 mM) at 37° C. for 30 min, then imaged with fluorescent microscopy. Example fluorescence micrographs are shown in FIG. 7. The majority of cells seeded on fibrin electrospun scaffolds were alive, as measured by live/dead staining (>98% cell viability). Likewise, the high number of live cells at day 10 indicated that the PRF electrospun scaffold was mechanically stable and appeared to actively promote adhesion, proliferation, and migration of the cells, as shown in FIGS. 7A and 7B.

In particular, these figures illustrate the cell viability of primary bovine satellite cells seeded on the surface of fibrin microfibers fabricated by electrospinning. The cells were cultured for 10 days in standard cell culture conditions. Calcein-AM was used as a fluorescent biomarker for cell viability using fluorescent microscopy.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, [symbol] and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in [symbol] “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A cultivated meat product, comprising:
microcarriers comprising fibrin; and
non-human animal cells.

2-85. (canceled)

86. The cultivated meat product of claim 1, wherein the microcarriers are edible.

87. The cultivated meat product of claim 1, wherein the microcarriers are biodegradable.

88. The cultivated meat product of claim 1, wherein the microcarriers comprise non-human blood plasma.

89. The cultivated meat product of claim 88, wherein the non-human blood plasma comprises a fibrinogen.

90. The cultivated meat product of claim 1, wherein the animal cells comprise myoblasts.

91. The cultivated meat product of claim 1, wherein the animal cells comprise fibroblasts.

92. The cultivated meat product of claim 1, wherein the animal cells comprise stem cells.

93. The cultivated meat product of claim 92, wherein the stem cells comprise mesenchymal stem cells.

94. The cultivated meat product of claim 92, wherein the stem cells comprise embryonic stem cells.

95. The cultivated meat product of claim 92, wherein the stem cells comprise bone marrow derived stem cells.

96. The cultivated meat product of claim 92, wherein the stem cells comprise adipose-derived stem cells.

97. A method, comprising:
fabricating microcarriers comprising a hydrogel comprising fibrin; and
culturing non-human cells on the microcarriers.

98. The method of claim 97, comprising culturing the cells in cell growth medium.

99. The method of claim 98, wherein the cell growth medium comprises a platelet rich plasma.

100. The method of claim 98, wherein the cell growth medium comprises serum.

101. The method of claim 100, wherein the serum comprises non-human animal serum.

102. The method of claim 97, wherein the cells comprise non-human muscle cells.

103. A method, comprising:
lysing non-human red blood cells to produce a cell lysate; and
mixing the cell lysate and non-human muscle cells on microcarriers comprising fibrin to produce a tissue mass of at least 10 g.

104. The method of claim 103, further comprising lysing the non-human red blood cells within 24 hours of withdrawal of the non-human red blood cells from a non-human living donor.
